(19) United States
(12) Patent Application Publication
(10) Pub. No.: US 2009/0299418 A1
(43) Pub. Date: Dec. 3, 2009

(54) CONCURRENT BILATERAL SPG MODULATION

(75) Inventors: Alon Shalev, Ra'anana (IL); Rinat Borenshtain, Holon (IL); Itschak Lamensdorf, Modiin (IL)

Correspondence Address: DARBY & DARBY P.C., P.O. BOX 770, Church Street Station, New York, NY, 10008-0770, US

(73) Assignee: Brainsgate LTD., Ra'anana (IL)

(21) Appl. No.: 11/573,993

(22) PCT Filed: Aug. 23, 2005
(86) PCT No.: PCT/IL05/00912

§ 371 Date: Jun. 1, 2009

Related U.S. Application Data
(60) Provisional application No. 60/709,734, filed on Aug. 19, 2005.
(60) Provisional application No. 60/604,037, filed on Aug. 23, 2004.

(51) Int. Cl. A61N 1/05; A61M 37/00

(52) U.S. Cl. 607/3; 604/20

(57) ABSTRACT

Apparatus (1) is provided, including a first electrode (7a) and a second electrode (7b), adapted to be applied to a first site and a second site of a subject, respectively, the first site different from the second site, and the first and second sites selected from the list consisting of: a left sphenopalatine ganglion (SPG) (6a), a right SPG (6b), a left vidian nerve, a right vidian nerve, a left greater palatine nerve, a right greater palatine nerve, a left lesser palatine nerve, a right lesser palatine nerve, a left sphenopalatine nerve, a right sphenopalatine nerve, a left otic ganglion, a right otic ganglion, an afferent fiber going into the left otic ganglion, an afferent fiber going into the right otic ganglion, an efferent fiber going out of the left otic ganglion, and an efferent fiber going out of the right otic ganglion. A control unit (8) is adapted to drive a current that travels in sequence from the control unit (8) to the first electrode (7a), to the first site, to the second site, to the second electrode (7b), and back to the control unit (8).

### CROSS-REFERENCES TO RELATED APPLICATIONS

This application claims priority from:

(a) U.S. Provisional Patent Application 60/604,037 to Shalev et al., filed Aug. 23, 2004, entitled, “Concurrent bilateral SPG modulation”; and

(b) a U.S. provisional patent application to Dayan et al., filed Aug. 19, 2005, entitled, “Stimulation for treating brain events and other conditions.”

Both of these applications are assigned to the assignee of the present application and are incorporated herein by reference.

### FIELD OF THE INVENTION

The present invention relates generally to medical procedures and devices. More specifically, the invention relates to the use of electrical stimulation for treating medical conditions.

### BACKGROUND OF THE INVENTION

The blood-brain barrier (BBB) is a unique feature of the central nervous system (CNS) which isolates the brain from the systemic blood circulation. To maintain the homeostasis of the CNS, the BBB prevents access to the brain of many substances circulating in the blood.

PCT Patent Publication WO 01/85094 to Shalev and Gross, which is assigned to the assignee of the present patent application and is incorporated herein by reference, describes apparatus for modifying a property of a brain of a patient, including electrodes applied to a sphenopalatine ganglion (SPG) or a neural tract originating in or leading to the SPG. A control unit drives the electrodes to apply a current capable of inducing (a) an increase in permeability of a blood-brain barrier (BBB) of the patient, (b) a change in cerebral blood flow of the patient, and/or (c) an inhibition of parasympathetic activity of the SPG.

U.S. Pat. No. 6,853,858 to Shalev, which is assigned to the assignee of the present application and is incorporated herein by reference, describes apparatus for delivering a Non Steroidal Anti-Inflammatory Drug (NSAID) supplied to a body of a subject for delivery to at least a portion of a central nervous system (CNS) of the subject via a systemic blood circulation of the subject. The apparatus includes a stimulator adapted to stimulate at least one site of the subject, so as to cause an increase in passage of the NSAID from the systemic blood circulation across a blood brain barrier (BBB) of the subject to the portion of the CNS, during at least a portion of the time that the NSAID is present in the blood, the site selected from the list consisting of: a sphenopalatine ganglion (SPG), an anterior ethmoidal nerve, a posterior ethmoidal nerve, a communicating branch between an anterior ethmoidal nerve and a retro-orbital branch of an SPG, a communicating branch between a posterior ethmoidal nerve and a retro-orbital branch of an SPG, a greater palatine nerve, a lesser palatine nerve, a sphenopalatine nerve, a communicating branch between a maxillary nerve and an SPG, a nasopalatine nerve, a posterior nasal nerve, an infraorbital nerve, an otic ganglion, an afferent fiber going into the otic ganglion, an efferent fiber going out of the otic ganglion, a vidian nerve, a greater superficial petrosal nerve, and a lesser deep petrosal nerve.

U.S. Pat. No. 6,526,318 to Ansarinia and related PCT Publication WO 01/97905 to Ansarinia, which are incorporated herein by reference, describe a method for the suppression or prevention of various medical conditions, including pain, movement disorders, autonomic disorders, and neuropsychiatric disorders. The method includes positioning an electrode on or proximate to at least one of the patient's SPG, sphenopalatine nerves, or vidian nerves, and activating the electrode to apply an electrical signal to such nerve. In a further embodiment for treating the same conditions, the electrode used is activated to dispense a medication solution or analgesic to such nerve. The '318 patent and '905 publication also describe surgical techniques for implanting the electrode.

U.S. Pat. No. 6,405,079 to Ansarinia, which is incorporated herein by reference, describes a method for the suppression or prevention of various medical conditions, including pain, movement disorders, autonomic disorders, and neuropsychiatric disorders. The method includes positioning an electrode adjacent to or around a sinus, the dura adjacent a sinus, or falx cerebri, and activating the electrode to apply an electrical signal to the site. In a further embodiment for treating the same conditions, the electrode dispenses a medication solution or analgesic to the site. The '079 patent also describes surgical techniques for implanting the electrode.

U.S. Pat. No. 6,432,986 to Levin and PCT Publication WO 99/03473 to Levin, which are incorporated herein by reference, describe techniques for inhibiting a cerebral neurovascular disorder or a muscular headache. The techniques include intranasally administering a pharmaceutical composition comprising a long-acting local anesthetic.

U.S. Pat. No. 6,491,940 to Levin, US Patent Application 2003/0133877 to Levin, and PCT Publication WO 00/44432 to Levin, which are incorporated herein by reference, describe techniques for inhibiting a cerebral neurovascular disorder or a muscular headache. The techniques include intranasally administering a pharmaceutical composition comprising a long-acting local anesthetic. Apparatus for delivering or applying the composition is also described.

US Patent Application 2001/0004644 to Levin and PCT Publication WO 01/43733 to Levin, which are incorporated herein by reference, describe techniques for inhibiting cephalic inflammation, including meningeal inflammation and cerebral inflammation. The techniques include intranasally administering a long-acting local anesthetic. Apparatus for delivering or applying the composition is also described, including a dorsonasally implanted electronic neural stimulator, such as a transepithelial neural stimulation device.

The following patent application publications, all of which are assigned to the assignee of the present application and are incorporated herein by reference, may be of interest: WO 03/090599, WO 03/105658, WO 04/010923, WO 04/043218, WO 04/044947, WO 04/045242, WO 04/043217, WO 04/043334, WO 05/030025, WO 05/030118, and US 2004/0220644.

The following patents and patent application publications, all of which are incorporated herein by reference, may be of interest: U.S. Pat. No. 5,756,071 to Mattern et al., U.S. Pat. No. 5,752,515 to Jolesz et al., U.S. Pat. Nos. 5,725,471 and 6,086,525 to Davey et al., PCT Publication WO 02/32504 to Zanger et al., US Patent Application Publication 2003/0050527 to Fox et al., U.S. Pat. No. 6,415,184 to Ishikawa et al., PCT Publications WO 03/084591, WO 03/020350, WO 03/000310, WO 02/068031, and WO 02/068029 to Djupesland, US Patent Application Publication 2003/0079742 to Giroux, and U.S. Pat. No. 5,855,907 to Peyman.

U.S. Pat. No. 4,886,493 to Yee, which is incorporated herein by reference, describes an applicator and process for accomplishing SPG block, including using an extended tube of a fixed length and width.

An article entitled “Endoscopic transnasal neurolytic sphenopalatine ganglion block for head and neck cancer pain,” by Varghese et al., J Laryngol Otol. 2001 May; 115(5):385-7, which is incorporated herein by reference, describes nasal endoscopy as a valuable adjunct to the localization of the sphenopalatine ganglion. Twenty-two patients with advanced malignancies of the head and neck region whose pain was not adequately controlled with conventional medications, including oral morphine, were given nasal endoscopically-guided neurolytic sphenopalatine ganglion block with six per cent phenol, after a prognostic block with local anesthetic solution. Seventeen patients had good immediate relief. One had partial relief and four had inadequate relief. On follow-up for one month, the patients had significantly lower pain intensity and the pain was more manageable with oral medication.

The following references, which are incorporated herein by reference, may be useful:
Delepine L, Aubineau P, “Plasma protein extravasation induced in the rat dura mater by stimulation of the parasympathetic sphenopalatine ganglion,” Experimental Neurology, 147, 389-400 (1997)
Hara H, Zhang Q J, Kuroyanagi T, Kobayashi S, “Parasympathetic cerebrovascular innervation: An anterograde tracing from the sphenopalatine ganglion in the rat,” Neurosurgery, 32, 822-827 (1993)
Jolliet-Riant P, Tillement J P, “Drug transfer across the blood-brain barrier and improvement of brain delivery,” Fundam. Clin. Pharmacol., 13, 16-25 (1999)
Kroll R A, Neuwelt E A, “Outwitting the blood brain barrier for therapeutic purposes: Osmotic opening and other means,” Neurosurgery, 42, 1083-1100 (1998)
Sanders M, Zuurmond W W, “Efficacy of sphenopalatine ganglion blockade in 66 patients suffering from cluster headache: A 12-70 month follow-up evaluation,” Journal of Neurosurgery, 87, 876-880 (1997)
Syelaz J, Hara H, Pinard E, Mraovitch S, MacKenzie E T, Edvinsson L, “Effects of stimulation of the sphenopalatine ganglion on cortical blood flow in the rat,” Journal of Cerebral Blood Flow and Metabolism,” 8, 875-878 (1988)
Van de Waterbeemd H, Camenisch G, Folkers G, Chretien J R, Raevsky O A, “Estimation of blood brain barrier crossing of drugs using molecular size and shape and h bonding descriptors,” Journal of Drug Targeting,” 6, 151-165, (1998)
Suzuki N, Hardebo J E, Kahrstrom J, Owman C, “Selective electrical stimulation of postganglionic cerebrovascular parasympathetic nerve fibers originating from the sphenopalatine ganglion enhances cortical blood flow in the rat,” Journal of Cerebral Blood Flow and Metabolism, 10, 383-391 (1990)
Suzuki N, Hardebo J E, Kahrstrom J, Owman C H, “Effect on cortical blood flow of electrical stimulation of trigeminal cerebrovascular nerve fibres in the rat,” Acta Physiol. Scand., 138, 307-315 (1990)
Major A, Silver W, “Odorants presented to the rat nasal cavity increase cortical blood flow,” Chem. Senses, 24, 665-669 (1999)
Fusco B M, Fiore G, Gallo F, Martelletti P, Giacovazzo M, “‘Capsaicin-sensitive’ sensory neurons in cluster headache: pathophysiological aspects and therapeutic indications,” Headache, 34, 132-137 (1994)
Lambert G A, Bogduk N, Goadsby P J, Duckworth J W, Lance J W, “Decreased carotid arterial resistance in cats in response to trigeminal stimulation,” Journal of Neurosurgery, 61, 307-315 (1984)
Silver W L, “Neural and pharmacological basis for nasal irritation,” in Tucker W G, Leaderer B P, Mølhave L, Cain W S (eds), Sources of Indoor Air Contaminants, Ann. NY Acad. Sci., 641, 152-163 (1992)
Silver W, “Chemesthesis: the burning questions,” ChemoSense, Vol. 2, 1-2 (1999)
Devoghel J C, “Cluster headache and sphenopalatine block,” Acta Anaesthesiol Belg., 32(1):101-7 (1981)
Branston N M, “The physiology of the cerebrovascular parasympathetic innervation,” British Journal of Neurosurgery 9:319-329 (1995)
Branston N M et al., “Contribution of cerebrovascular parasympathetic and sensory innervation to the short-term control of blood flow in rat cerebral cortex,” J Cereb Blood Flow Metab 15(3):525-31 (1995)
Toda N et al., “Cerebral vasodilation induced by stimulation of the pterygopalatine ganglion and greater petrosal nerve in anesthetized monkeys,” Neuroscience 96(2):393-398 (2000)
Seylaz J et al., “Effect of stimulation of the sphenopalatine ganglion on cortical blood flow in the rat,” J Cereb Blood Flow Metab 8(6):875-8 (1988)
Nollet H et al., “Transcranial magnetic stimulation: review of the technique, basic principles and applications,” The Veterinary Journal 166:28-42 (2003)
Van Gijn J et al., “Subarachnoid haemorrhage: diagnosis, causes and management,” Brain 124:249-278 (2001)
Goadsby P J et al., “Effect of stimulation of trigeminal ganglion on regional cerebral blood flow in cats,” Am J Physiol 22:R270-R274 (1987)
Walters B B et al., “Cerebrovascular projections from the sphenopalatine and otic ganglia to the middle cerebral artery of the cat,” Stroke 17:488-494 (1986)
Suzuki N et al., “Trigeminal fibre collaterals storing substance P and calcitonin gene-related peptide associate with ganglion cells containing choline acetyltransferase and vasoactive intestinal polypeptide in the sphenopalatine ganglion of the rat. An axon reflex modulating parasympathetic ganglionic activity?” Neuroscience 30:595-604 (1989)
Roth B J et al., “In vitro evaluation of a 4-leaf coil design for magnetic stimulation of peripheral nerve,” Electroencephalography and Clinical Neurophysiology 93:68-74 (1994)
Hotta H et al., “Effects of stimulating the nucleus basalis of Meynert on blood flow and delayed neuronal death following transient ischemia in rat cerebral cortes,” Jap J Phys 52:383-393 (2002)
Reis D J et al., “Electrical stimulation of cerebellar fastigial nucleus reduces ischemic infarction elicited by middle cerebral artery occlusion in rat,” J Cereb Blood Flow Metab 11(5):810-8 (1991)
Matsui T et al., “The effects of cervical spinal cord stimulation (cSCS) on experimental stroke,” Pacing Clin Electrophysiol 12(4 Pt 2):726-32 (1989)
Segher O et al., “Spinal cord stimulation reducing infract volume in model of focal cerebral ischemia in rats,” J Neurosurg 99(1):131-137 (2003)
Zhang R et al., “A nitric oxide donor induces neurogenesis and reduces functional deficits after stroke in rats,” Ann Neurol 50:602-611 (2001)
Ziche M et al., “Nitric oxide and angiogenesis,” J Neurooncol 50:139-148 (2000)
Kawamata T et al., “Intracisternal basic fibroblast growth factor (bFGF) enhances behavioral recovery following focal cerebral infarction in the rat,” J Cereb Blood Flow Metab 16:542-547 (1996)
Zhang Z G et el., “VEGF enhances angiogenesis and promotes blood-brain barrier leakage in the ischemic brain,” J Clin Invest 106:829-838 (2000)
Sun Y et al., “Neuronal nitric oxide synthase and ischemia-induced neurogenesis,” J Cereb Blood Flow Metab 25(4):485-92 (2005)
Zhang F et al., “Nitric oxide donors increase blood flow and reduce brain damage in focal ischemia: evidence that nitric oxide is beneficial in the early stages of cerebral ischemia,” J Cereb Blood Flow Metab 14(2):217-26 (1994)
Beridze M et al., “Effect of nitric oxide initial blood levels on erythrocyte aggregability during 12 hours from ischemic stroke onset,” Clin Hemorheol Microcirc 30(3-4):403-6 (2004)
Davis S M et al., “Advances in penumbra imaging with MR,” Cerebrovasc Dis 17 Suppl 3:23-7 (2004)
Phan T G et al., “Salvaging the ischaemic penumbra: more than just reperfusion?” Clin Exp Pharmacol Physiol 29(1-2):1-10 (2002)
Gressens P et al., “Neuroprotection of the developing brain by systemic administration of vasoactive intestinal peptide derivatives,” J Pharmacol Exp Ther 288 (3):1207-13 (1999)
Zhang R et al., “Nitric oxide enhances angiogenesis via the synthesis of vascular endothelial growth factor and cGMP after stroke in the rat,” Circ Res 21; 92(3):308-13 (2003)
de la Torre J C, “Vascular basis of Alzheimer's pathogenesis,” Ann NY Acad Sci 977:196-215 (2002)
Roman G C, “Cholinergic dysfunction in vascular dementia,” Curr Psychiatry Rep 7(1):18-26 (2005)
Tony J F L, “Nitric oxide and the cerebral vascular function,” J Biomed Sci 7:16-26 (2000)
Pluta R M, “Delayed cerebral vasospasm and nitric oxide: review, new hypothesis, and proposed treatment,” Pharmacol Ther 105(1):23-56 (2005)
Sandgren K et al., “Vasoactive intestinal peptide and nitric oxide promote survival of adult rat myenteric neurons in culture,” J Neurosci Res 72(5):595-602 (2003)
Laude K et al., “NO produced by endothelial NO synthase is a mediator of delayed preconditioning-induced endothelial protection,” Am J Physiol Heart Circ Physiol 284(6):H2053-60 (2003) (Epub 2003 Jan. 9)
Khan M et al., “S-Nitrosoglutathione reduces inflammation and protects brain against focal cerebral ischemia in a rat model of experimental stroke,” J Cereb Blood Flow Metab 25(2):177-92 (2005)
Molloy J et al., “S-nitrosoglutathione reduces the rate of embolization in humans,” Circulation 98(14):1372-5 (1998)
Schmid-Elsaesser R et al., “A critical reevaluation of the intraluminal thread model of focal cerebral ischemia. Evidence of inadvertent premature reperfusion and subarachnoid hemorrhage in rats by laser-Doppler flowmetry,” Stroke 29:2162-2170 (1998)
Zausinger V S et al., “Neurological impairment in rats after transient middle cerebral artery occlusion: a comparative study under various treatment paradigms,” Brain Research 863(1-2):94-105 (2000)
Hunter A J et al., “To what extent have functional studies of ischemia in animals been useful in the assessment of potential neuroprotective agents?” Trends Pharmacol Sci 19:59-66 (1998)
Varghese et al., “Endoscopic transnasal neurolytic sphenopalatine ganglion block for head and neck cancer pain,” J Laryngol Otol 115(5):385-7 (2001)
Kanner A A et al., “Serum S100beta: a noninvasive marker of blood-brain barrier function and brain lesions,” Cancer 97(11):2806-13 (2003)

### SUMMARY OF THE INVENTION

In embodiments of the present invention, an electrical stimulator comprises first and second electrodes, adapted to contact or to be positioned in a vicinity of a first sphenopalatine ganglion (SPG) and a second SPG, respectively, of a patient. The stimulator is configured to simultaneously apply a positive voltage and a negative voltage to the first and second electrodes, respectively, so as to modulate both. SPGs. The stimulator modulates the SPGs in order to control and/or modify SPG-related behavior, e.g., in order to induce changes in cerebral blood flow and/or to modulate permeability of the blood-brain barrier (BBB). These embodiments may be used in many medical applications, such as, by way of illustration and not limitation, (a) the treatment of cerebrovascular disorders such as stroke, (b) the facilitation of drug transport across the BBB, (c) the facilitation of a diagnosis of a condition of the central nervous system (CNS), (d) the facilitation of delivery of diagnostic molecules across the BBB, (e) the facilitation of delivery of a biotechnological product or another therapeutic moiety that does not cross the intact BBB, or (f) the treatment of migraine, cluster and other types of headaches.

As used herein, in the context of stimulation of a nerve structure, the word “stimulation” (and variants thereof), includes both excitation and inhibition of the nerve structure.

For some applications, the electrodes are alternatively or additionally adapted to be applied to a pair of one of the following “modulation target sites” (MTS):
a nerve of the pterygoid canal (also called a vidian nerve), such as a greater superficial petrosal nerve (a preganglionic parasympathetic nerve) or a lesser deep petrosal nerve (a postganglionic sympathetic nerve);
a greater palatine nerve;
a lesser palatine nerve;
a sphenopalatine nerve;
an otic ganglion;
an afferent fiber going into the otic ganglion; or
an efferent fiber going out of the otic ganglion.

It is to be appreciated that implantation and modulation sites, methods of implantation, and parameters of modulation are described herein by way of illustration and not limitation, and that the scope of the present invention includes other possibilities which would be obvious to someone of ordinary skill in the art who has read the present patent application.

It is additionally to be appreciated that whereas some embodiments of the present invention are described with respect to application of electrical currents to tissue, this is to be understood in the context of the present patent application and in the claims as being substantially equivalent to applying an electrical field, e.g., by creating a voltage drop between two electrodes.

There is therefore provided, in accordance with an embodiment of the present invention, apparatus including:

a first electrode and a second electrode, adapted to be applied to a first site and a second site of a subject, respectively, the first site different from the second site, and the first and second sites selected from the list consisting of: a left sphenopalatine ganglion (SPG), a right SPG, a left vidian nerve, a right vidian nerve, a left greater palatine nerve, a right greater palatine nerve, a left lesser palatine nerve, a right lesser palatine nerve, a left sphenopalatine nerve, a right sphenopalatine nerve, a left otic ganglion, a right otic ganglion, an afferent fiber going into the left otic ganglion, an afferent fiber going into the right otic ganglion, an efferent fiber going out of the left otic ganglion, and an efferent fiber going out of the right otic ganglion; and

a control unit, adapted to drive a current that travels in sequence from the control unit to the first electrode, to the first site, to the second site, to the second electrode, and back to the control unit.

In an embodiment, the control unit is adapted to configure the current to increase a permeability of a blood-brain barrier (BBB) of both hemispheres of a brain of the subject. Alternatively, the control unit is adapted to configure the current to increase the permeability of the BBB of a single hemisphere of the brain. In an embodiment, the control unit is adapted to configure the current to induce a change in cerebral blood flow (CBF) in both hemispheres of a brain of the subject. Alternatively, the control unit is adapted to configure the current to induce the change in CBF in a single hemisphere of the brain. In an embodiment, the control unit is adapted to configure the current to induce an increase in a release a substance in both hemispheres of a brain of the subject, the substance selected from the list consisting of: a neuroprotective substance, and a neurorestorative substance. Alternatively, the control unit is adapted to configure the current to induce the increase in the release of the substance in a single hemisphere of the brain.

In an embodiment, the first site is contralateral to the second site, and the first and second electrodes are adapted to be applied to the contralateral first and second sites, respectively. For some applications, the control unit is adapted to configure the current to induce a greater increase in permeability of a BBB of a target hemisphere of a brain of the subject than of a BBB of the other hemisphere of the brain. Alternatively or additionally, the control unit is adapted to configure the current to induce a greater increase in CBF in a target hemisphere of a brain of the subject than in the other hemisphere of the brain. Further alternatively or additionally, the control unit is adapted to configure the current to induce a greater increase in release of at least one substance in a target hemisphere of a brain of the subject than in the other hemisphere of the brain, the substance selected from the list consisting of: a neuroprotective substance, and a neurorestorative substance.

For some applications, at least one of the first and second electrodes is monopolar. For some applications, the apparatus includes a connecting element, coupled to the first and second electrodes, and adapted to be passed through at least a portion of a greater palatine canal of the subject.

In an embodiment, the first site is ipsilateral to the second site, and the first and second electrodes are adapted to be applied to the ipsilateral first and second sites, respectively.

In an embodiment, the first and second sites include the left and right SPGs, respectively, and the first and second electrodes are adapted to be applied to the left and right SPGs, respectively. Alternatively, the first and second sites include the right and left SPGs, respectively, and the first and second electrodes are adapted to be applied to the right and left SPGs, respectively.

There is further provided, in accordance with an embodiment of the present invention, a method including driving a current from a first site of a subject to a second site of the subject different from the first site, the first and second sites selected from the list consisting of: a left sphenopalatine ganglion (SPG), a right SPG, a left vidian nerve, a right vidian nerve, a left greater palatine nerve, a right greater palatine nerve, a left lesser palatine nerve, a right lesser palatine nerve, a left sphenopalatine nerve, a right sphenopalatine nerve, a left otic ganglion, a right otic ganglion, an afferent fiber going into the left otic ganglion, an afferent fiber going into the right otic ganglion, an efferent fiber going out of the left otic ganglion, and an efferent fiber going out of the right otic ganglion.

In an embodiment, the second site is contralateral to the first site, and driving the current includes driving the current from first site to the contralateral second site. For some applications, the method includes administering, to a systemic circulation of the subject, a therapeutic compound selected to treat a condition of a target hemisphere of a brain of the subject, and driving the current includes configuring the current to induce a greater increase in transport of the compound from the systemic circulation, across a BBB of the target hemisphere, and into the target hemisphere, than across a BBB of the other hemisphere of the brain, and into the other hemisphere. Alternatively or additionally, the method includes selecting a target hemisphere of a brain of the subject that has experienced a brain event, and driving the current includes configuring the current to induce a greater increase in CBF in the target hemisphere than in the other hemisphere of the brain. Further alternatively or additionally, the method includes selecting a target hemisphere of a brain of the subject that has experienced a brain event, and driving the current includes configuring the current to induce a greater increase in a release of at least one substance in the target hemisphere than in the other hemisphere of the brain, the substance selected from the list consisting of: a neuroprotective substance, and a neurorestorative substance.

For some applications, driving the current includes passing a stimulation device through at least a portion of a greater palatine canal of the subject, and driving the current from the device.

The present invention will be more fully understood from the following detailed description of the embodiments thereof, taken together with the drawings, in which:

### BRIEF DESCRIPTION OF THE DRAWINGS

FIG. 1 is a schematic illustration of a fully-implantable electrical stimulation system, in accordance with an embodiment of the present invention;

FIG. 2 is a schematic pictorial view of an implantation configuration of the stimulation system of FIG. 1, in accordance with an embodiment of the present invention;

FIG. 3 is a schematic pictorial view of another implantation configuration of the stimulation system of FIG. 1, in accordance with an embodiment of the present invention; and

FIG. 4 is a bar graph showing experimental data collected in accordance with an embodiment of the present invention.

### DETAILED DESCRIPTION OF THE INVENTION

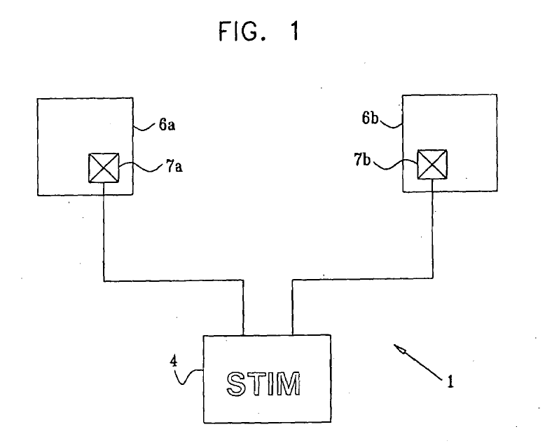

FIG. 1 is a schematic illustration of a fully-implantable electrical stimulation system 1, for simultaneous stimulation of a first sphenopalatine ganglion (SPG) 6a and a second SPG 6b of a patient, in accordance with an embodiment of the present invention. Stimulation system 1 comprises an electrical stimulator 4, and at least a first electrode 7a and a second electrode 7b, which are adapted to contact or be positioned in a vicinity of first SPG 6a and second SPG 6b, respectively. Electrodes 7a and 7b are typically monopolar. Stimulator 4 is configured to simultaneously apply a positive voltage to one of the electrodes, and a negative voltage to the other electrode, so as to modulate both SPGs 6a and 6b. For some applications, stimulator 4 (e.g., a control unit thereof) is adapted to be capable of reversing the direction of the applied voltage, such that one of the electrodes serves as the anode during a portion of a stimulation session, and as the cathode during another portion of the session, and/or such that the direction of the applied voltage is selectable after stimulation system 1 has been implanted.

Stimulator 4 modulates the SPGs in order to control and/or modify SPG-related behavior, e.g., in order to induce changes in cerebral blood flow and/or to modulate permeability of the blood-brain barrier (BBB). Such stimulation may be used in many medical applications, such as, by way of illustration and not limitation, (a) the treatment of cerebrovascular disorders such as stroke, (b) the facilitation of drug transport across the BBB, (c) the facilitation of a diagnosis of a condition of the central nervous system (CNS), (d) the facilitation of delivery of diagnostic molecules across the BBB, (e) the facilitation of delivery of a biotechnological product or another therapeutic moiety that does not cross the intact BBB, or (f) the treatment of migraine, cluster and other types of headaches. Such stimulation may also be performed in conjunction with techniques described in the patents, patent application publications, and articles incorporated herein by reference.

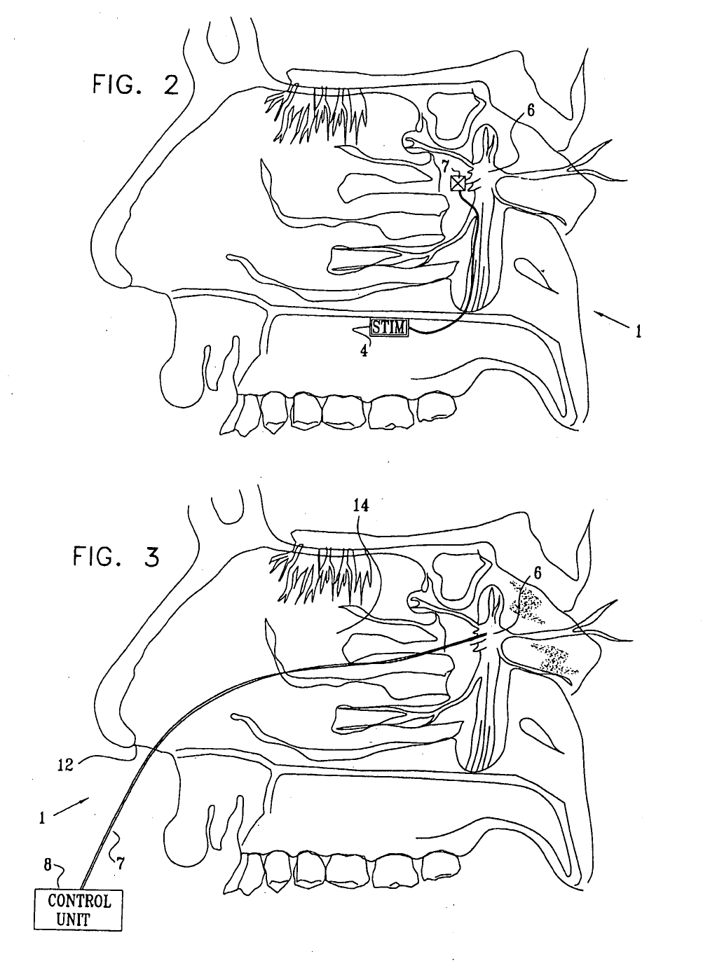

FIG. 2 is a schematic pictorial side view of stimulator 4 implanted between the hard palate and the mucoperiosteum (not shown) of the roof of the mouth, in accordance with an embodiment of the present invention. Because the figure is in side view, only one of electrodes 7a and 7b (labeled with the numeral 7) and one of SPGs 6a and 6b (labeled with the numeral 6) are shown.

For some applications, stimulator 4 is implanted on top of the bony palate, in the bottom of the nasal cavity. Alternatively or additionally, the stimulator is implanted at the lower side of the bony palate, at the top of the oral cavity. In this instance, flexible electrodes 7 are passed through the palatine bone or posterior to the soft palate, so as to be in a position to stimulate the SPG. Further alternatively or additionally, the stimulator may be directly attached to the SPG.

For some applications, stimulator 4 is delivered to a desired point within the nasal cavity by removably attaching stimulator 4 to the distal end of a rigid or slightly flexible introducer rod (not shown) and inserting the rod into one of the patient's nasal passages until the stimulator is properly positioned. As appropriate, the placement process may be facilitated by fluoroscopy, x-ray guidance, fine endoscopic surgery (FES) techniques or by any other effective guidance method known in the art, or by combinations of the aforementioned. Preferably, the ambient temperature and/or cerebral blood flow is measured concurrently with insertion. The cerebral blood flow may be measured with, for example, a laser Doppler unit positioned at the patient's forehead or transcranial Doppler measurements. Verification of proper implantation of the electrodes onto the appropriate neural structure may be performed by activating the device, and generally simultaneously monitoring cerebral blood flow, and/or monitoring sensations reported by the patient, such as paresthesias in the nose.

The placement process may be performed using techniques described in U.S. Provisional Patent Application 60/426,180 filed Nov. 14, 2002, entitled, “Surgical tools and techniques for stimulation,” PCT Application PCT/IL 2003/000966, filed Nov. 13, 2003, of the same title, which claims priority from the '180 application, and/or a US application filed May 11, 2005, in the national stage thereof. All of these applications are assigned to the assignee of the present patent application and are incorporated herein by reference.

The passage of certain molecules from cerebral blood vessels into the brain is hindered by the BBB. The endothelium of the capillaries, the plasma membrane of the blood vessels, and the foot processes of the astrocytes all impede uptake by the brain of the molecules. The BBB generally allows only small molecules (e.g., hydrophilic molecules of molecular weight less than about 400 Da, and lipophilic molecules of less than about 500 Da) to pass from the circulation into the brain.

As used in the present application and in the claims, the BBB comprises the tight junctions opposing the passage of most ions and large molecular weight compounds between the blood and tissue in the brain, such as tissue of the brain or tumor tissue.

In accordance with an embodiment of the present invention, parasympathetic activation induced by current from stimulator 4 overcomes the resistance to trans-BBB molecular movement generated by the endothelium of the cerebral capillaries and the plasma membrane, and/or increases permeability via other mechanisms, such as by increasing transcytosis. For some applications, therefore, stimulator 4 may be used to transiently remove a substantial obstacle to the passage of diagnostic and/or therapeutic agents from the systemic blood circulation to the CNS, and/or of biochemical agents from the CNS to the systemic blood circulation.

It is hypothesized that at least two neuromodulators play an important role in this change in properties of the BBB—vasoactive intestinal polypeptide (VIP) and nitric oxide (NO). (Acetylcholine may also be involved.) VIP is a short peptide, and NO is a gaseous molecule. VIP and NO are believed to be major factors in facilitating plasma protein extravasation (PPE). For some applications, stimulator 4 is adapted to vary parameters of the current applied to SPGs or MTSs, as appropriate, in order to selectively influence the activity of one or both of these neuromodulators. For example, stimulation of the parasympathetic nerve at different frequencies can induce differential secretion—low frequencies cause secretion of NO, while high frequencies (e.g., above about 10 Hz) cause secretion of peptides (VIP).

FIG. 3 is a schematic illustration of a stimulator control unit 8 of stimulation system 1 positioned external to a patient's body, in accordance with an embodiment of the present invention. At least two flexible electrodes 7a and 7b extend from control unit 8, through a nostril 12 of the patient, and to positions within a nasal cavity 14 that are adjacent to SPGs 6. Because the figure is in side view, only one of electrodes 7a and 7b (labeled with the numeral 7) and one of SPGs 6a and 6b (labeled with the numeral 6) are shown.

Each of electrodes 7a and 7b typically comprises a suitable conductive material, for example, a physiologically-acceptable material such as silver, iridium, platinum, a platinum iridium alloy, titanium, nitinol, or a nickel-chrome alloy. For some applications, one or more of the electrodes have lengths ranging from about 1 to 5 mm, and diameters ranging from about 50 to 100 microns. Each electrode is preferably insulated with a physiologically-acceptable material such as polyethylene, polyurethane, or a co-polymer of either of these. The electrodes are preferably spiral in shape, for better contact, and may have a hook shaped distal end for hooking into or near the SPG. Alternatively or additionally, the electrodes may comprise simple wire electrodes, spring-loaded “crocodile” electrodes, or adhesive probes, as appropriate.

In an embodiment of the invention, each of electrodes 7a and 7b comprises a substantially smooth surface, except that the distal end of each such electrode is configured or treated to have a large surface area. For example, the distal tip may be porous platinized. Alternatively or additionally, at least the tip of electrodes 7a and 7b, and/or a metal housing of stimulator 4 includes a coating comprising an anti-inflammatory drug, such as beclomethasone sodium phosphate or beclomethasone phosphate. Alternatively, such an anti-inflammatory drug is injected or otherwise applied.

Typically, a determination regarding whether to use a configuration such as that shown in FIG. 2 or that shown in FIG. 3 is made responsive to a frequency or total number of procedures anticipated. When this frequency or total number is high, the preference is for a configuration such as that shown in FIG. 2, while one-time or infrequent procedures indicates for a configuration such as that shown in FIG. 3.

In an embodiment of the present invention, electrodes 7a and 7b are alternatively or additionally adapted to be applied to two of the MTSs, as defined hereinabove, or to one of the SPGs and one of the MTSs. For some applications, the electrodes are applied to a pair of one of the MTSs that are anatomically symmetrical (i.e., a left and right particular MTS), while for other applications, the electrodes are applied to two different MTSs, either contralaterally or ipsilaterally to each other.

As used in the specification and in the claims, stimulation of both SPGs 6a and 6b or a pair of MTSs to facilitate transport of a diagnostic agent from the systemic blood circulation to the CNS, is to be understood as including stimulation prior to, during, and/or after administration of the agent to the systemic circulation. For patients in whom a stimulator previously was implanted for therapeutic purposes, such implanted stimulator may be used for performing stimulation to facilitate a diagnosis, as described herein.

In an embodiment of the present invention, stimulation of both SPGs 6a and 6b or a pair of MTSs is configured to increase the transport of a diagnostic agent across the BBB from a non-CNS tissue, such as the systemic blood circulation, into the CNS. The diagnostic agent is typically administered to the systemic blood circulation, such as intravenously, and a diagnostic procedure, typically an imaging modality, is then performed directly on the CNS. For some applications, the diagnostic agent comprises a tracer agent, such as an imaging contrast agent, for example, a Magnetic Resonance Imaging (MRI) contrast agent, a Single Photon Emission Computed Tomography (SPECT) radioisotope, a Positron Emission Tomography (PET) radioisotope, an ultrasound contrast enhancer, or an X-ray contrast agent (e.g., for a Computerized Tomography (CT) or angiography imaging sequence).

In an embodiment of the present invention, stimulation of both SPGs 6a and 6b or a pair of MTSs is configured to increase the transport of a biochemical agent across the BBB from the CNS to a non-CNS tissue, such as the systemic blood circulation. Such biochemical agents are typically disease-specific biochemical markers. Prior to stimulation of an MTS to increase BBB permeability, the concentration of such a biochemical agent is typically greater in the CNS than in the systemic circulation, i.e., there is a concentration gradient across the endothelium. Therefore, increasing the permeability of the BBB, typically acutely, generally releases the agent into the systemic circulation. Once in the systemic circulation, diagnosis is typically performed by sampling a body tissue or fluid, typically blood, and analyzing the whole blood, plasma, or serum. Analysis is typically performed using a biochemical assay or another analytical procedure, such as imaging, in order to qualitatively or quantitatively probe the presence of the biochemical agent of interest, a metabolite thereof, or a chemical or biological derivative thereof.

“Diagnosis,” as used in the present patent application, including the claims, is to be understood as comprising the art or act of recognizing the presence of disease from its signs or symptoms, deciding as to the character (e.g., stage) of a disease, screening for disease, and/or predicting the onset of disease. Diagnosis may be performed in vivo or in vitro, as appropriate. Diagnosis may comprise a combination of diagnostic procedures. For example, the permeability of the BBB may be increased in combination with taking a blood sample and analyzing it for the presence of a biochemical marker of a CNS neoplastic process, and performing PET imaging for a mAb or pAb to a protein that is indicative of a neoplastic process.

The functioning BBB inhibits clearance of neurotoxic compounds, such as β-Amyloid, from the CNS into the systemic circulation. These potentially neurotoxic compounds are therefore not metabolized and removed from the body to the extent desired, and therefore continue to have undesired effects in the CNS. In an embodiment of the present invention, stimulation of both SPGs 6a and 6b or a pair of MTSs is configured to increase clearance of neurotoxic compounds, such as β-Amyloid, from the CNS into the systemic circulation. Once in the systemic circulation, these neurotoxic compounds may be metabolized and removed from the body with greater ease and with fewer side effects, compared to effects that accompany their presence in the CNS.

In an embodiment of the present invention, stimulation of both SPGs 6a and 6b or a pair of MTSs is configured to increase transport of a drug from the systemic circulation across the BBB into the CNS. These techniques may be combined with techniques described in one or more of the applications cited hereinbelow.

In an embodiment of the present invention, both SPGs 6a and 6b or a pair of MTSs are electrically stimulated using one or more of the following stimulation parameters:
The total duration of stimulation is between about 0.25 and about 4 hours, such as about 3 hours.
Stimulation is applied with a duty cycle (intermittency) of about 5 minutes “active stimulation,” and about 10 minutes “withholding from stimulation.” (The active stimulation period is typically between about 2 and about 10 minutes, while the withholding from stimulation period is typically between about 5 and about 15 minutes.)
During the active stimulation period, stimulation is applied for an “on” period of between about 30 and about 90 seconds of each successive period within the active stimulation period, and, thereafter, not applied during an “off” period, for between about 30 and about 60 seconds of the total period.
During the “on” periods, stimulation is applied as repeated pulses having a pulse width of between about 250 and about 1000 microseconds, each typically followed by a duration of sufficient length to enable repolarization of nerve tissue of the MTS, e.g., about 99 ms. These example values represent an effective 10 Hz signal. Other suitable values range from about 2 Hz to about 30 Hz.
Each pulse typically has a magnitude less than about 8 V, such as between about 1 and about 7 V, for example, about 3.5 V. The current of the pulse is between about 0.2 and about 10 mA, such as between about 0.5 and about 5 mA, for example, between about 1 and about 2 mA.

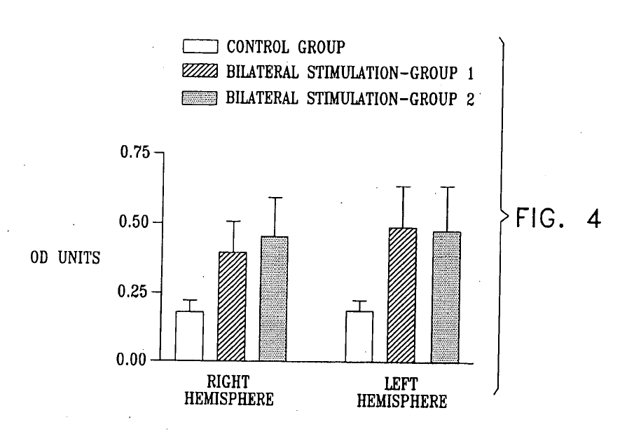

FIG. 4 is a bar graph showing experimental data collected in accordance with an embodiment of the present invention. Bilateral SPG stimulation was performed on two groups of mice: Group 1 included 6 ICR mice, and Group 2 included 7 C57/BL mice. A third group of 6 mice served as a control. The mice were anesthetized with Pental 60 mg/kg. A custom-made bipolar hook electrode was implanted such that one side of the electrode was near the right ethmoidal nerve and the other side of the electrode was near the left ethmoidal nerve, in the vicinity of the right and left SPGs, respectively, of each mouse. The mice of the control group were anesthetized and operated upon, but no electrodes were implanted. Proper placement of the electrodes was confirmed by verifying the response to stimulation, such as mild tremor and response of the eye lids, which were found earlier to correlate with SPG stimulation in small rodent species.

Prior to stimulation (and in the control group), 2 ml/kg of Evans blue (2%) solution at 35° C. was administered intravenously to the femoral vein. Stimulation was applied bilaterally to the SPGs of the mice, using the following parameters: a stimulation duration of 60 minutes, including alternating “on” periods of 90 seconds followed by “off” periods of 60 seconds. During each “on” period, pulses of amplitude 5 Volts were applied, each pulse having a pulse width of 1 millisecond. The pulses were separated by 99 milliseconds (i.e., the applied pulse frequency was 10 Hz).

After stimulation (and in the control group), the brains were harvested, divided into left and right hemispheres, and weighed. Dimethylformamide (Sigma) (×2 volume/weight) was added, the brain tissue was ground and centrifuged, and supernatant fluid was collected. The quantity of Evans blue in the supernatant fluid was measured using 630 nm UV light absorbance.

The graph in FIG. 4 shows the results obtained in Group 1, Group 2, and the control group. The x-axis represents the right and left brain hemispheres in the three groups, and the y-axis represents the absorption of Evans blue in the hemispheres, expressed in optical density (OD) units. Error bars indicate standard error. The results obtained demonstrate an average 2.5-fold increase in the penetration of Evans blue to both hemispheres of the mouse brain in the experimental groups vs. the control group.

In an embodiment of the present invention, stimulation of both SPGs 6a and 6b or a pair of contralateral MTSs is performed in order to treat a condition of a single target hemisphere of a brain of the subject. For some applications, a therapeutic compound is administered to a systemic circulation of the subject, and the stimulation is configured to induce a greater increase in transport of the compound from the systemic circulation, across a BBB of the target hemisphere, and into the target hemisphere, than across a BBB of the other hemisphere of the brain, and into the other hemisphere of the brain. Alternatively or additionally, the condition includes a brain event, such as an ischemic event (e.g., a stroke), and the stimulation is configured to induce a greater increase in CBF in the target hemisphere than in the other hemisphere, and/or to induce a greater increase in the release of one or more neuroprotective substances, such as neuromodulators (e.g., nitric oxide (NO) and/or vasoactive intestinal polypeptide (VIP)), and/or one or more neurorestorative substances, in the target hemisphere than in the other hemisphere, so as to treat the brain event.

In an embodiment of the present invention, stimulation of both SPGs 6a and 6b or a pair of MTSs is configured to induce an increase in permeability of a BBB of both hemispheres of a brain of the subject, to induce a change in CBF in both hemispheres, and/or to induce an increase in a release, in both hemispheres, of a substance, such as a neuroprotective substance and/or a neurorestorative substance.

The scope of the present invention includes embodiments described in the following applications, which are assigned to the assignee of the present application and are incorporated herein by reference. In an embodiment, techniques described in this application are practiced in combination with methods and apparatus described in one or more of the following patent applications:
U.S. Provisional Patent Application 60/203,172, filed May 8, 2000, entitled, “Method and apparatus for stimulating the sphenopalatine ganglion to modify properties of the BBB and cerebral blood flow”
U.S. Provisional Patent Application 60/364,451, filed Mar. 15, 2002, entitled, “Applications of stimulating the sphenopalatine ganglion (SPG)”
U.S. Provisional Patent Application 60/368,657, filed Mar. 28, 2002, entitled, “SPG Stimulation”
U.S. Provisional Patent Application 60/376,048, filed Apr. 25, 2002, entitled, “Methods and apparatus for modifying properties of the BBB and cerebral circulation by using the neuroexcitatory and/or neuroinhibitory effects of odorants on nerves in the head”
U.S. Provisional Patent Application 60/388,931, filed Jun. 14, 2002, entitled “Methods and systems for management of Alzheimer's disease,” PCT Patent Application PCT/IL03/000508, filed Jun. 13, 2003, claiming priority therefrom, and a US patent application filed Dec. 14, 2004 in the national stage thereof
U.S. Provisional Patent Application 60/400,167, filed Jul. 31, 2002, PCT Patent Application PCT/IL03/000631, filed Jul. 31, 2003, entitled, “Delivering compounds to the brain by modifying properties of the BBB and cerebral circulation,” and a US patent application filed Jan. 31, 2005 in the national stage thereof
U.S. patent application Ser. No. 10/258,714, filed Oct. 25, 2002, entitled, “Method and apparatus for stimulating the sphenopalatine ganglion to modify properties of the BBB and cerebral blood flow,” or the above-referenced PCT Publication WO 01/85094
U.S. Provisional Patent Application 60/426,180, filed Nov. 14, 2002, entitled, “Surgical tools and techniques for sphenopalatine ganglion stimulation,” PCT Patent Application PCT/IL03/000966, filed Nov. 13, 2003, which claims priority therefrom, and a US patent application filed May 11, 2005 in the national stage thereof
U.S. Provisional Patent Application 60/426,182, filed Nov. 14, 2002, and a corresponding PCT application claiming priority therefrom, filed Nov. 13, 2003, entitled, “Stimulation circuitry and control of electronic medical device,” and a US patent application filed May 11, 2005 in the national stage thereof
U.S. patent application Ser. No. 10/294,310, filed Nov. 14, 2002, entitled, “SPG stimulation for treating eye pathologies,” and PCT Patent Application PCT/IL03/000965, filed Nov. 13, 2003, claiming priority therefrom
U.S. patent application Ser. No. 10/294,343, filed Nov. 14, 2002, which issued as U.S. Pat. No. 6,853,858 to Shalev, and a corresponding PCT application claiming priority therefrom, filed Nov. 13, 2003, entitled, “Administration of anti-inflammatory drugs into the CNS”
U.S. Provisional Patent Application 60/426,181, filed Nov. 14, 2002, entitled, “Stimulation for treating ear pathologies,” PCT Patent Application PCT/IL03/000963, filed Nov. 13, 2003, which claims priority therefrom, and a US patent application filed May 11, 2005 in the national stage thereof
U.S. Provisional Patent Application 60/448,807, filed Feb. 20, 2003, entitled, “Stimulation for treating autoimmune-related disorders of the CNS”
U.S. Provisional Patent Application 60/461,232 to Gross et al., filed Apr. 8, 2003, entitled, “Treating abnormal conditions of the mind and body by modifying properties of the blood-brain barrier and cephalic blood flow”
PCT Patent Application PCT/IL03/00338 to Shalev, filed Apr. 25, 2003, entitled, “Methods and apparatus for modifying properties of the BBB and cerebral circulation by using the neuroexcitatory and/or neuroinhibitory effects of odorants on nerves in the head,” and U.S. patent application Ser. No. 10/512,780, filed Oct. 25, 2004 in the national stage thereof
U.S. Provisional Patent Application 60/506,165, filed Sep. 26, 2003, entitled, “Diagnostic applications of stimulation”
U.S. patent application Ser. No. 10/678,730, filed Oct. 2, 2003, entitled, “Targeted release of nitric oxide in the brain circulation for opening the BBB,” and PCT Patent Application PCT/IL04/000911, filed Oct. 3, 2004, claiming priority therefrom
PCT Patent Application PCT/ IL04/000897, filed Sep. 26, 2004, entitled, “Stimulation for treating and diagnosing conditions”
U.S. patent application Ser. No. 10/952,536, filed Sep. 27, 2004, entitled, “Stimulation for treating and diagnosing conditions”
U.S. patent application Ser. No. 10/783,113, filed Feb. 20, 2004, entitled, “Stimulation for acute conditions”
a US provisional patent application filed Aug. 19, 2005, entitled, “Stimulation for treating brain events and other conditions”

By way of example and not limitation, stimulation system 1 may utilize circuitry described in one or more of these patent applications.

It is to be understood that whereas some embodiments of the present invention are described hereinabove with respect to applying a voltage drop between the left and right SPGs (e.g., by means of pulses applied through electrodes applied to each SPG), the scope of the present invention includes simultaneously applying a field to both SPGs with respect to a common ground electrode, or alternating between application of a signal to one SPG and application of a signal to the contralateral SPG.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description. For example, elements which are shown in a figure to be housed within one integral unit may, for some applications, be disposed in a plurality of distinct units. Similarly, apparatus for communication and power transmission which are shown to be coupled in a wireless fashion may be, alternatively, be coupled in a wired fashion, and apparatus for communication and power transmission which are shown to be coupled in a wired fashion may be, alternatively, be coupled in a wireless fashion.

1. Apparatus comprising:
a first electrode and a second electrode, adapted to be applied to a first site and a second site of a subject, respectively, the first site different from the second site, and the first and second sites selected from the list consisting of: a left sphenopalatine ganglion (SPG), a right SPG, a left vidian nerve, a right vidian nerve, a left greater palatine nerve, a right greater palatine nerve, a left lesser palatine nerve, a right lesser palatine nerve, a left sphenopalatine nerve, a right sphenopalatine nerve, a left otic ganglion, a right otic ganglion, an afferent fiber going into the left otic ganglion, an afferent fiber going into the right otic ganglion, an efferent fiber going out of the left otic ganglion, and an efferent fiber going out of the right otic ganglion; and
a control unit, adapted to drive a current that travels in sequence from the control unit to the first electrode, to the first site, to the second site, to the second electrode, and back to the control unit.

2. The apparatus according to claim 1, wherein the control unit is adapted to configure the current to increase a permeability of a blood-brain barrier (BBB) of both hemispheres of a brain of the subject.

3. The apparatus according to claim 1, wherein the control unit is adapted to configure the current to induce a change in cerebral blood flow (CBF) in both hemispheres of a brain of the subject.

4. The apparatus according to claim 1, wherein the control unit is adapted to configure the current to induce an increase in a release a substance in both hemispheres of a brain of the subject, the substance selected from the list consisting of: a neuroprotective substance, and a neurorestorative substance.

5. The apparatus according to claim 1, wherein the first site is contralateral to the second site, and wherein the first and second electrodes are adapted to be applied to the contralateral first and second sites, respectively.

6. The apparatus according to claim 5, wherein the control unit is adapted to configure the current to induce a greater increase in permeability of a BBB of a target hemisphere of a brain of the subject than of a BBB of the other hemisphere of the brain.

7. The apparatus according to claim 5, wherein the control unit is adapted to configure the current to induce a greater increase in CBF in a target hemisphere of a brain of the subject than in the other hemisphere of the brain.

8. The apparatus according to claim 5, wherein the control unit is adapted to configure the current to induce a greater increase in release of at least one substance in a target hemisphere of a brain of the subject than in the other hemisphere of the brain, the substance selected from the list consisting of: a neuroprotective substance, and a neurorestorative substance.

9. The apparatus according to claim 1, wherein at least one of the first and second electrodes is monopolar.

10. The apparatus according to claim 1, comprising a connecting element, coupled to the first and second electrodes, and adapted to be passed through at least a portion of a greater palatine canal of the subject.

11. The apparatus according to claim 1, wherein the first site is ipsilateral to the second site, and wherein the first and second electrodes are adapted to be applied to the ipsilateral first and second sites, respectively.

12. The apparatus according to claim 1, wherein the first and second sites include the left and right SPGs, respectively, and wherein the first and second electrodes are adapted to be applied to the left and right SPGs, respectively.

13. The apparatus according to claim 1, wherein the first and second sites include the right and left SPGs, respectively, and wherein the first and second electrodes are adapted to be applied to the right and left SPGs, respectively.

14. A method comprising driving a current from a first site of a subject to a second site of the subject different from the first site, the first and second sites selected from the list consisting of: a left sphenopalatine ganglion (SPG), a right SPG, a left vidian nerve, a right vidian nerve, a left greater palatine nerve, a right greater palatine nerve, a left lesser palatine nerve, a right lesser palatine nerve, a left sphenopalatine nerve, a right sphenopalatine nerve, a left otic ganglion, a right otic ganglion, an afferent fiber going into the left otic ganglion, an afferent fiber going into the right otic ganglion, an efferent fiber going out of the left otic ganglion, and an efferent fiber going out of the right otic ganglion.

15. The method according to claim 14, wherein driving the current comprises configuring the current to induce an increase in a permeability of a blood-brain barrier (BBB) of both hemispheres of a brain of the subject.

16. The method according to claim 14, wherein driving the current comprises configuring the current to induce a change in cerebral blood flow (CBF) in both hemispheres of a brain of the subject.

17. The method according to claim 14, wherein driving the current comprises configuring the current to induce an increase in a release a substance in both hemispheres of a brain of the subject, the substance selected from the list consisting of: a neuroprotective substance, and a neurorestorative substance.

18. The method according to claim 14, wherein the second site is contralateral to the first site, and wherein driving the current comprises driving the current from first site to the contralateral second site.

19. The method according to claim 18, comprising administering, to a systemic circulation of the subject, a therapeutic compound selected to treat a condition of a target hemisphere of a brain of the subject, wherein driving the current comprises configuring the current to induce a greater increase in transport of the compound from the systemic circulation, across a BBB of the target hemisphere, and into the target hemisphere, than across a BBB of the other hemisphere of the brain, and into the other hemisphere.

20. The method according to claim 18, comprising selecting a target hemisphere of a brain of the subject that has experienced a brain event, wherein driving the current comprises configuring the current to induce a greater increase in CBF in the target hemisphere than in the other hemisphere of the brain.

21. The method according to claim 18, comprising selecting a target hemisphere of a brain of the subject that has experienced a brain event, wherein driving the current comprises configuring the current to induce a greater increase in a release of at least one substance in the target hemisphere than in the other hemisphere of the brain, the substance selected from the list consisting of: a neuroprotective substance, and a neurorestorative substance.

22. The method according to claim 14, wherein driving the current comprises passing a stimulation device through at least a portion of a greater palatine canal of the subject, and driving the current from the device.

23. The method according to claim 14, wherein the second site is ipsilateral to the first site, and wherein driving the current comprises driving the current from first site to the ipsilateral second site.

24. The method according to claim 14, wherein the first and second sites include the left and right SPGs, respectively, and wherein driving the current comprises driving the current from the left SPG to the right SPG.

25. The method according to claim 14, wherein the first and second sites include the right and left SPGs, respectively, and wherein driving the current comprises driving the current from the right SPG to the left SPG.
